(19) United States
(12) Patent Application Publication
(10) Pub. No.: US 2013/0158417 A1
(43) Pub. Date: Jun. 20, 2013

(54) METHOD, APPARATUS AND COMPUTER PROGRAM FOR AUTOMATIC NON-INVASIVE BLOOD PRESSURE MEASUREMENT

(71) Applicant: Alle Borger, Helsinki (FI)

(72) Inventor: Alle Borger, Helsinki (FI)

(73) Assignee: GENERAL ELECTRIC COMPANY, Schenectady, NY (US)

(21) Appl. No.: 13/328,422

(22) Filed: Dec. 16, 2011

(51) Int. Cl. A61B 5/021

(52) U.S. Cl. 600/485

(57) ABSTRACT

A method, apparatus and computer program product are disclosed for non-invasively determining blood pressure of a subject. To improve the specificity of automatic blood pressure determinations in a patient monitor provided with a non-invasive blood pressure determination unit, a physiological index indicative of sympathetic activity is derived from a subject, variations in the physiological index are monitored, and the blood pressure determination unit is instructed to initiate blood pressure determination when the variations fulfill a predetermined condition.

### BACKGROUND OF THE INVENTION

This disclosure relates generally to automatic activation of non-invasive blood pressure measurement.

Blood pressure measurements may be divided into non-invasive and invasive measurement methods. An invasive blood pressure measurement is carried out with intravascular cannulae by placing the needle of the cannulae in an artery. Invasive measurement may used when continuous tracking of blood pressure is required and when accurate information about the waveform of blood pressure is required. However, invasive blood pressure measurements have some inherent drawbacks, which include the risk of infection, thrombosis, damage of the vessel wall, and bleeding. Therefore, patients with invasive blood pressure monitoring require more work and supervision than patients that do not require invasive measurement. Furthermore, non-invasive measurements are simpler to carry out and require less training of the nursing staff. Therefore, an invasive measurement is often used only if an accurate or reliable insight of blood pressure cannot be obtained through non-invasive measurement methods.

A traditional non-invasive blood pressure measurement employs a stethoscope, an inflatable cuff, and a pressure manometer. As the traditional method requires a skilled clinician to carry out the measurement, it is suitable for non-recurring spot-checks but not for constant monitoring of blood pressure. Therefore, various automatically activated non-invasive blood pressure measurement mechanisms have been developed, which activate the blood pressure cuff if a sensor signal obtained from the patient indicates that there may be a change in the blood pressure of the patient.

In one known mechanism, heart rate variability (HRV) is evaluated and blood pressure measurement is activated if a significant change is detected in the HRV. In another known mechanism, the user may set a fixed measurement interval time between two regular blood pressure measurements and the apparatus employs a plethysmographic signal obtained from the subject to detect whether a need to measure the blood pressure arises during the fixed measurement interval time between two successive regular blood pressure measurements. The plethysmographic signal obtained at a regular blood pressure measurement is stored and used as reference data with which the plethysmographic signal obtained on-line from the subject is compared. If a significant change is detected in the on-line plethysmographic signal with respect to the reference data, the device may determine that the subject's blood pressure has changed since the latest regular measurement and may trigger a new blood pressure measurement.

A major drawback related to the automatic non-invasive blood pressure measurements is that the physiological sensor data, based on which the decision is made to activate the measurement, is not strongly related to the actual physiological mechanisms that regulate blood pressure. That is, there is no one-to-one correspondence between changes in the physiological sensor data used for the decision-making, such as plethysmographic data or HRV data, and changes in the blood pressure. This is because various other factors than blood pressure may affect the physiological sensor data. For example, changes in the amplitude of the plethysmographic signal may be due to vasoconstriction or vasodilation, which may be caused by certain medications, for example, while HRV may be affected by hormones, temperature, sleep-wake cycle, and stress.

Consequently, the measurement decisions have a rather low specificity with respect to the blood pressure changes and therefore the cuff is often pressurized although there is no significant change in the blood pressure. Frequent pressurizations may lead to tissue damages and unnecessary pressurizations may also be disturbing in view of the care process. This is the case in a sleep laboratory, for example, where unnecessary disturbances are to be avoided during the sleep of the subject.

### BRIEF DESCRIPTION OF THE INVENTION

The above-mentioned problems are addressed herein which will be comprehended from the following specification.

The apparatus or system monitors variations in a physiological index indicative of the autonomic reactions that regulate blood pressure and if the variations fulfill a predetermined condition, blood pressure measurement is activated. Thus, blood pressure measurement is controlled by changes in the index. The index typically provides a fixed diagnostic scale whose readings are independent of the subject in question and therefore no subject-specific calibration is needed, but the measurement may be started without any calibration for the subject in question.

In an embodiment, a method for non-invasively determining blood pressure in a patient monitor comprises providing the patient monitor with a non-invasive blood pressure determination unit and deriving a physiological index from a subject, wherein the physiological index is indicative of sympathetic activity in the subject. The method further comprises monitoring variations in the physiological index and instructing the blood pressure determination unit to initiate blood pressure determination when the variations fulfill a predetermined condition.

In another embodiment, an apparatus for non-invasively determining blood pressure of a subject comprises a blood pressure determination unit for non-invasively determining blood pressure of a subject, an index determination unit configured derive a physiological index from a subject, wherein the physiological index is indicative of sympathetic activity in the subject, and an index monitoring unit configured to monitor variations in the physiological index and to instruct the blood pressure determination unit to initiate blood pressure determination when the variations fulfill a predetermined condition.

In a still further embodiment, a computer program product for non-invasively determining blood pressure of a subject comprises a first program product portion configured to monitor variations in a physiological index indicative of sympathetic activity in a subject and to generate a start command for a blood pressure determination unit when the variations fulfill a predetermined condition, thereby to initiate blood pressure determination.

Various other features, objects, and advantages of the invention will be made apparent to those skilled in the art from the following detailed description and accompanying drawings.

### BRIEF DESCRIPTION OF THE DRAWINGS

FIG. 1 is a block diagram illustrating one embodiment of an apparatus/system provided with automatic, non-invasive blood pressure measurement;

FIG. 2 is a flow diagram illustrating an embodiment of the index determination used in connection with the automatic blood pressure measurement;

FIG. 3 illustrates a typical transform used in the index determination;

FIG. 4 is a flow diagram illustrating two embodiments of the index monitoring used in connection with automatic blood pressure measurement; and

FIG. 5 illustrates the entities of the apparatus/system in terms of the automatic, non-invasive blood pressure measurement.

### DETAILED DESCRIPTION OF THE INVENTION

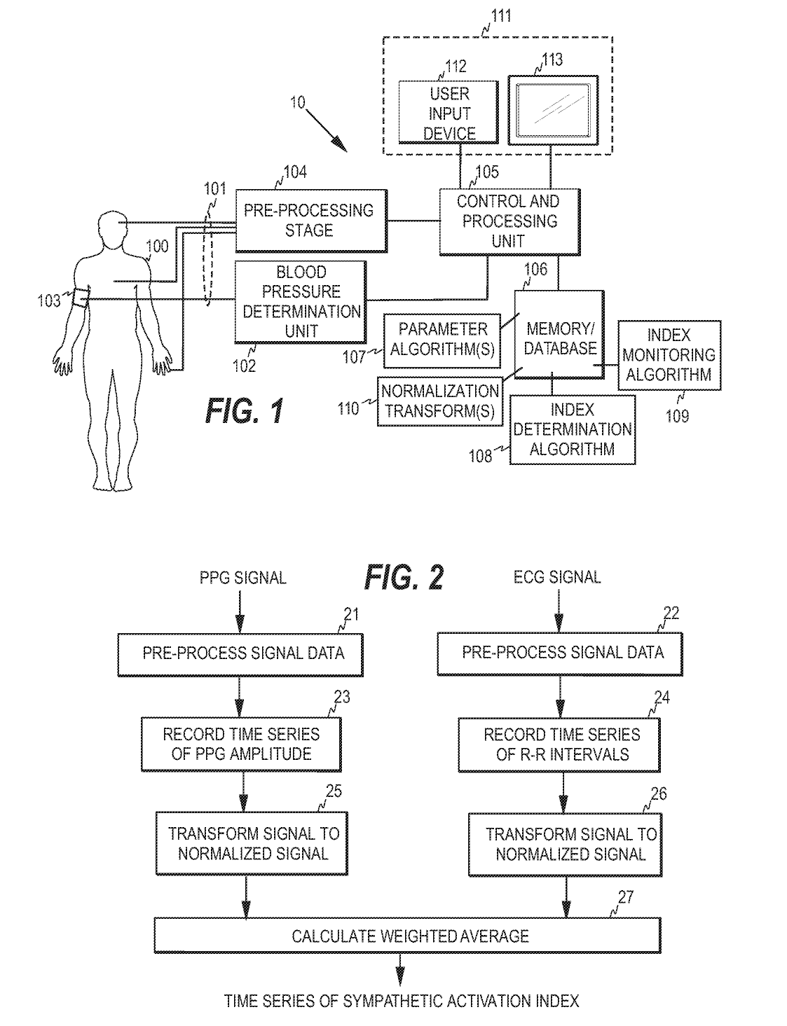

FIG. 1 illustrates one embodiment of a monitoring apparatus/system 10 for monitoring a subject 100. A monitoring apparatus/system normally acquires a plurality of physiological signals 101 from the subject, where one physiological signal corresponds to one measurement channel. The physiological signals may typically comprise several types of signals, such as ECG, EEG, blood pressure, respiration, and plethysmographic signals. Based on the raw real-time physiological signal data obtained from the subject, a plurality of physiological parameters may be determined. A physiological parameter here refers to a variable calculated from the waveform data of one or more channel signals acquired from the subject. The physiological parameter may also represent a waveform signal value determined over a predefined period of time, although the physiological parameter is typically a distinct parameter derived from one or more measurement channels. Each signal parameter may be assigned one or more alarm limits to alert the nursing staff when the parameter reaches or crosses the alarm limit.

The monitoring apparatus/system of FIG. 1 utilizes a standard non-invasive blood pressure (NIBP) measurement setup in the sense that the apparatus/system comprises a standard blood pressure determination unit 102 provided with a pressurizable cuff 103. The cuff 103 is placed in a normal manner around the arm of a subject 100 and the blood pressure determination unit controls the pressure of the cuff to obtain blood pressure data. Since the blood pressure determination unit is logically a separate unit in the monitoring apparatus/system, it is shown as a separate entity in FIG. 1. However, the unit may also be embedded into the apparatus/system.

Apart from the blood pressure signals, which may be processed in unit 102, the physiological channel signals acquired from the subject are supplied to a control and processing unit 105 through a pre-processing stage 104 comprising typically an input amplifier and a filter. The control and processing unit (or the pre-processing stage) converts the signals into digitized format for each measurement channel. The digitized signal data may then be stored in the memory 106 of the control and processing unit. The control and processing unit also receives blood pressure data from the blood pressure determination unit and sends trigger messages to the blood pressure determination unit to trigger blood pressure determination in the blood pressure determination unit.

For monitoring the subject, the control and processing unit is provided with one or more parameter algorithm(s) 107 configured to determine one or more physiological parameters, such as SpO₂ or pulse rate, from the subject. The control and processing unit is further provided with an index determination algorithm 108 and with an index monitoring algorithm 109. The index determination algorithm is configured to determine a physiological index indicative of the sympathetic activation of the autonomous nervous system (ANS) of the subject 100. The index monitoring algorithm is configured to monitor the behavior of the index and to initiate a blood pressure measurement if a significant change (rise) is detected in the index. The index is here termed sympathetic activation index since it is indicative of the sympathetical activation in the ANS, which also controls the blood pressure. In the determination of the index, the algorithm 108 may utilize normalization transforms 110 that may be stored in memory 106.

The monitoring apparatus/system of FIG. 1 further includes a user interface 111 including one or more user input devices 112, such as a keyboard, and one or more display units 113.

FIG. 2 illustrates one embodiment of the index determination algorithm, in which the index is determined based on two normalized signals. In this embodiment, the normalized signals are determined based on a photoplethysmographic (PPG) signal and an ECG signal. The measurement of the PPG and ECG signal waveform data may be implemented in a conventional manner, i.e. while the patient is connected to the patient monitoring system, the signal waveform data is recorded and stored in the memory of the apparatus/system. The PPG data may be obtained from a pulse oximeter sensor, while the ECG data may be obtained from ECG sensors. The recorded PPG and ECG waveform data may then be pre-processed in steps 21 and 22, respectively, for filtering out some of the frequency components of the respective signal or for rejecting artifacts, for example. These steps are not necessary, but may be performed to improve the quality of the signal data.

As to the PPG signal, the pulse amplitude of the waveform signal is extracted for each pulse beat in step 23, thereby to obtain a time series of the amplitude of the pulsative component of the peripheral blood circulation. As to the ECG signal, the R-R interval is derived from the ECG waveform for each pulse beat in step 24, thereby to obtain a time series of the R-R interval.

Each time series is then subjected to a normalization transform (steps 25 and 26) to obtain a time series of normalized PPG amplitude (PPGA) and a time series of a normalized R-R interval (RRI). The normalization transform here refers to a process that converts the input signal values to a predetermined output value range, such as 0 to 100.

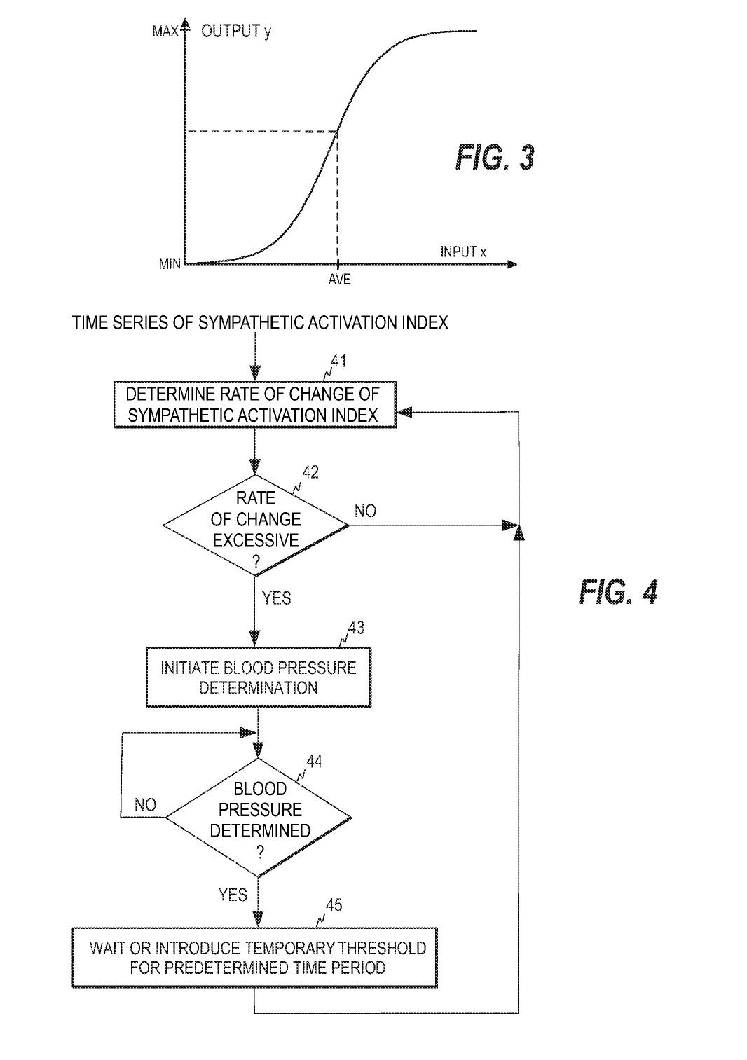

FIG. 3 illustrates typical input-output characteristics of the normalization transform. The curve of a typical function transform corresponds to a so-called sigmoid function, i.e. the output value y depends on the input value x according to equation (1):

$$y = \frac{A}{1 + {e}^{- B \times x}} , \qquad ( 1 )$$

where A and B are parameters. Parameter A is typically a positive constant determining the scale of the index values, while B may be a patient-specific parameter, which determines the distribution of the output index values within the scale from zero to A. As can be seen from FIG. 3, the transform forces the input signal to a predetermined output value range between a minimum value MIN and a maximum value MAX. For Eq. (1), MIN equals to 0, while MAX equals to A.

Each normalization transform may be a non-adaptive, partially adaptive, or fully adaptive normalization transform, which may be implemented as a parameterized transform or as a histogram transform. Adaptability here refers to the ability of the transform to adapt to the incoming data, i.e. to the data measured from the subject. In full or partial adaptation the transform is made dependent on signal data measured earlier from the subject in question, while in a non-adaptive transform the transform is implemented without adaptation to the incoming data. As the transform applied to the input signal is a normalization transform that typically depends on subject-specific history data, the input signal may be transformed to an index signal that provides a fixed diagnostic scale whose readings are independent of the subject in question. Therefore, the blood pressure measurement control is automatically ready for any subject without a calibration process.

With reference to FIG. 2 again, the normalized PPG amplitude and the normalized R-R interval are then combined in step 27 to form an aggregate indicator that forms the sympathetic activation index. This may be performed, for example, by calculating a weighted average of the two normalized values for each data point pair (PPGA/RRI) obtained from the two time series.

To give an example of preferred values of the two weights, the weighted average WA may be calculated for example as follows:

WA=100−(0.3×RRI(norm)+0.7×PPGA(norm)),

where RRI(norm) refers to the normalized R-R interval and PPGA(norm) to the normalized PPG amplitude. Step 27 thus outputs a time series of the weighted average.

The weighted average serves as the sympathetic activation index which is indicative of autonomic reaction, particularly of the sympathetical activation in the ANS. This type of an index is often available in a patient monitor since it is also indicative of surgical stress, i.e. balance between nociception and antinociception during surgery. Pain, discomfort, and surgical stress may activate the sympathetical branch of the ANS and cause an increase in blood pressure, heart rate and adrenal secretions, and the index indicates the balance between nociception (pain, discomfort, stress) and antinociception (blocking or suppression of nociception in the pain pathways at the subcortical level). As the index is indicative of the sympathetical activation, there is also a strong correlation between the index and the blood pressure, which means that changes in the index may be used to assess when a blood pressure determination should be initiated to check possible changes in the blood pressure.

FIG. 4 illustrates two embodiments of the index monitoring algorithm 109. The time series of the sympathetic activation index obtained from step 27 of algorithm 108 is supplied as input data to algorithm 109, which first determines the rate of change of the sympathetic activation index in step 41. The rate of change, i.e. the time derivative of the index, indicates the amount of change in a time unit. The obtained rate of change is compared with a predetermined gradient threshold in step 42 to check whether the rate of change has reached or exceeded the predetermined threshold value. If this is the case, the index monitoring algorithm initiates blood pressure determination by supplying a start command to the blood pressure determination unit 102 (step 43). In response to this, the blood pressure determination unit activates the cuff 103, performs a blood pressure measurement, and informs the control and processing unit of the result. When the index monitoring algorithm detects that the blood pressure determination is completed (step 44/yes), it may wait (step 45) for a predetermined time period before returning to step 41 to re-start the above process. This wait time may be used to prevent the blood pressure determinations from occurring too frequently. Alternatively, the index monitoring algorithm may introduce a temporary gradient threshold for a predetermined time period in step 45, so that there will be monitoring data available continuously. The temporary gradient threshold may be substantially greater than the normal threshold, such as two times the normal gradient threshold. Thus, in this embodiment the index monitoring algorithm replaces the gradient threshold by a temporary threshold and returns to step 41 without any waiting period. Both alternatives are shown in step 45 of FIG. 4. In a combined embodiment, re-initiating of the blood determination may be inhibited during the wait time, although the determination of the rate of change, and possibly also the comparison of step 42, may be continued.

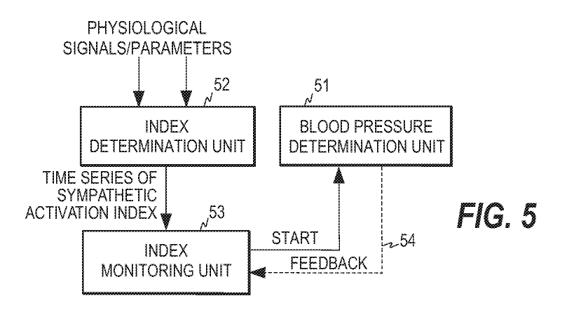

In terms of the determination of the blood pressure, the apparatus/system may be seen as an entity of three operational modules or units, as is illustrated in FIG. 5. A blood pressure determination unit 51 is configured to measure blood pressure non-invasively and an index determination unit 52 is configured to determine the time series of the sympathetic activation index. Further, an index monitoring unit 53 is configured to monitor variations in the index and to supply a start command to the blood pressure determination unit if the variations fulfill a predetermined condition. There may further be a feedback connection 54 from unit 51 to unit 53, which enables implementation of steps 44 and 45 to prevent too frequent cuff pressurizations. As discussed above, the index monitoring unit may utilize one or more gradient thresholds for preventing frequent cuff pressurizations. It is to be noted that FIG. 5 illustrates the division of the functionalities of the apparatus/system in logic sense and in view of the automatic blood pressure determination. In a real apparatus, the functionalities may be distributed in different ways between the elements or units of the apparatus/system.

As may be deduced from the description of FIGS. 1 and 5, a conventional monitoring apparatus/system 10 may be upgraded to enable the apparatus/system to determine blood related parameters in the above-described manner. Such an upgrade may be implemented, for example, by delivering to the apparatus/system a software module that enables the device to control the blood pressure determination in the above-described manner. The content of the software module may vary depending on the existing capabilities of the apparatus/system. If both the time series of the sympathetic activation index and the blood pressure determination unit are available in the apparatus/system, the software module may include the monitoring algorithm 109 only. The software module may be delivered, for example, on a data carrier, such as a CD or a memory card, or the through a telecommunications network.

In the above examples, the apparatus measures at least ECG and plethysmographic signals from the subject. However, the configuration of the monitoring apparatus/system 10 may vary depending on the type of the apparatus/system. That is, the above automatic blood pressure determination may be introduced into different types of patient monitors. For example, the apparatus of FIG. 1 may be a pulse oximeter, in which case only plethysmographic data is acquired from the subject. In a pulse oximeter, both the PPG amplitude and the R-R interval may be derived from the plethysmographic data. Furthermore, the index may be calculated based on different features or parameters indicative of activity in the sympathetic branch of the ANS, thereby to obtain the sympathetic activation index that controls the blood pressure determination. Such features/parameters include sympathovagal ratio, heart rate acceleration, and skin conductivity, for example. The number of parameters/features defining the index may also vary and a normalization transform may be applied to each parameter of the index.

The blood pressure determination unit may also be a separate unit or integrated with the monitoring apparatus or with the control and processing unit thereof. Instead of a separate blood pressure determination unit, the control and processing unit of FIG. 1 may thus be provided with a blood pressure determination algorithm adapted to control the cuff and to determine the blood pressure of the subject.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural or operational elements that do not differ from the literal language of the claims, or if they have structural or operational elements with insubstantial differences from the literal language of the claims.

1. A method for non-invasively determining blood pressure in a patient monitor, the method comprising:
providing a patient monitor with a non-invasive blood pressure determination unit;
deriving a physiological index from a subject, wherein the physiological index is indicative of sympathetic activity in the subject;
monitoring variations in the physiological index; and
instructing the blood pressure determination unit to initiate blood pressure determination when the variations fulfill a predetermined condition.

2. The method according to claim 1, wherein the deriving comprises determining a first physiological parameter and a second physiological parameter from at least one physiological signal acquired from the subject.

3. The method according to claim 2, wherein the deriving further comprises applying a first normalization transform to the first physiological parameter, thereby to obtain a first normalized physiological parameter and applying a second normalization transform to the second physiological parameter, thereby to obtain a second normalized physiological parameter.

4. The method according to claim 3, wherein the deriving further comprises calculating the physiological index as a weighted average of the first normalized physiological parameter and the second normalized physiological parameter.

5. The method according to claim 2, wherein the deriving comprises determining the first physiological parameter and the second physiological parameter, in which the first physiological parameter represents amplitude of a plethysmographic signal and the second physiological parameter represents heart beat interval.

6. The method according to claim 1, wherein the monitoring comprises
determining rate of change of the physiological index; and
comparing the rate of change with a predetermined threshold,
wherein the instructing comprises instructing the blood pressure determination unit to initiate the blood pressure determination when the rate of change reaches the predetermined threshold.

7. The method according to claim 6, wherein the monitoring further comprises temporarily replacing the predetermined threshold by a temporary threshold in response to the blood pressure determination.

8. The method according to claim 6, further comprising temporarily inhibiting the instructing in response to the blood pressure determination, thereby to control time interval between successive blood pressure determinations to exceed a predefined minimum length.

9. An apparatus for non-invasively determining blood pressure of a subject, the apparatus comprising:
a blood pressure determination unit for non-invasively determining blood pressure of a subject;
an index determination unit configured derive a physiological index from a subject, wherein the physiological index is indicative of sympathetic activity in the subject; and
an index monitoring unit configured to monitor variations in the physiological index and to instruct the blood pressure determination unit to initiate blood pressure determination when the variations fulfill a predetermined condition.

10. The apparatus according to claim 9, wherein the index determination unit is configured to determine a first physiological parameter and a second physiological parameter from at least one physiological signal acquired from the subject.

11. The apparatus according to claim 10, wherein the index determination unit is further configured to apply a first normalization transform to the first physiological parameter, thereby to obtain a first normalized physiological parameter and a second normalization transform to the second physiological parameter, thereby to obtain a second normalized physiological parameter.

12. The apparatus according to claim 11, wherein the index determination unit is further configured to calculate the physiological index as a weighted average of the first normalized physiological parameter and the second normalized physiological parameter.

13. The apparatus according to claim 10, wherein the first physiological parameter represents amplitude of a plethysmographic signal and the second physiological parameter represents heart beat interval.

14. The apparatus according to claim 9, wherein the index monitoring unit is configured to
determine rate of change of the physiological index;
compare the rate of change with a predetermined threshold; and
instruct the blood pressure determination unit to initiate the blood pressure determination when the rate of change reaches the predetermined threshold.

15. The apparatus according to claim 14, wherein the index monitoring unit is further configured to temporarily replace the predetermined threshold by a temporary threshold in response to the blood pressure determination.

16. The apparatus according to claim 9, wherein the index monitoring unit is adapted to temporarily inhibit control of the blood pressure determination unit in response to the blood pressure determination, thereby to control time interval between successive blood pressure determinations to exceed a predefined minimum length.

17. A computer program product for non-invasively determining blood pressure of a subject, the computer program product comprising a first program product portion configured to
monitor variations in a physiological index indicative of sympathetic activity in a subject; and
generate a start command for a blood pressure determination unit when the variations fulfill a predetermined condition, thereby to initiate blood pressure determination.

18. The computer program according to claim 17, further comprising a second program product portion adapted to determine the physiological index.
